(19) United States
(12) Patent Application Publication
(10) Pub. No.: US 2020/0406058 A1
(43) Pub. Date: Dec. 31, 2020

(54) Vascular Access Device

(71) Applicant: Photonx Holdings, Inc., Oldsmar, FL (US)

(72) Inventor: Scot Johnson, Tampa, FL (US)

(21) Appl. No.: 16/914,240

(22) Filed: Jun. 26, 2020

Related U.S. Application Data
(60) Provisional application No. 62/866,742, filed on Jun. 26, 2019.

(51) Int. Cl. A61N 5/06; A61M 39/02

(52) U.S. Cl. CPC A61N 5/0624; A61N 5/0601; A61M 39/0247; A61N 2005/0661; A61N 2005/067; A61N 2005/0662; A61N 2005/063; A61N 2005/0651; A61N 2005/0654; A61N 2005/0659

(57) ABSTRACT

A system for irradiation of a vascular space and its contents is presented. An adapter device having a vascular access end and an optical interface end can include a waveguide affixed within a waveguide lumen and extending outwardly through the vascular access end. The adapter device enables the simultaneous administration of radiation and exogenous fluids to a patient while maintaining the optical interface isolated from any fluids.

### FIELD OF THE INVENTION

The present invention generally relates to devices and methods for treatment of human blood. More particularly, the present invention relates to devices and methods for irradiating human blood in vivo.

### SUMMARY OF THE INVENTION

The present disclosure relates to an apparatus and method for treating blood in vivo using photonic infusion. Certain wavelengths of electromagnetic radiation, such as ultraviolet light, have the ability to affect biological and chemical structures. For example, the formation of thymine dimers under the influence of ultraviolet light is well known and has been utilized to sterilize surfaces by killing or inactivating a variety of pathogens.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The foregoing disclosure will be best understood and advantages thereof made most clearly apparent when consideration is given to the following detailed description in combination with the drawing figures presented. The detailed description makes reference to the following drawings:

FIG. 1 shows a patient receiving photonic infusion therapy;

FIG. 2 shows a three-dimensional view of the vascular access device according to the present disclosure; and

FIG. 3 shows the vascular access device of FIG. 2 in an exploded view;

FIG. 4A shows the interface of the vascular access device in a three-dimensional view;

FIG. 4B shows the interface of FIG. 4A from a top view;

FIG. 4C shows the interface of FIG. 4A from a right side view;

FIG. 4D shows the interface of FIG. 4A from a bottom view;

FIG. 4E shows the interface of FIG. 4A from a left end view;

FIG. 4F shows the interface of FIG. 4A from a right end view;

FIG. 5A shows the adapter housing of the vascular access device in a three-dimensional view;

FIG. 5B shows the adapter housing of FIG. 5A from a top view;

FIG. 5C shows the adapter housing of FIG. 5A from a side view;

FIG. 5D shows the adapter housing of FIG. 5A from a left end view; and

FIG. 5E shows the adapter housing of FIG. 5A from a right end view.

### DETAILED DESCRIPTION

Throughout the following discussion, numerous references will be made regarding sources of electromagnetic radiation. It should be appreciated that the use of such terms is deemed to represent one or more sources configured to produce electromagnetic energy, particularly ultraviolet, visible, and/or infrared light. Such light may be coherent or incoherent. For example, a source of electromagnetic energy can include one or more of an incandescent light, a metal vapor lamp, an HID lamp, a fluorescent lamp, a laser, a gas laser, an LED laser, a light emitting diode, and/or any suitable light source. Such sources of electromagnetic energy can be configured to produce a plurality of different wavelengths, and can also include devices for distribution of electromagnetic energy (for example, fiber optic cables and their associated connectors). It should also be appreciated that such sources may utilize a variety of optical connectors, for example an SMA-905 optical fiber connector. A device of the inventive concept can be compatible with any suitable optical connector.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

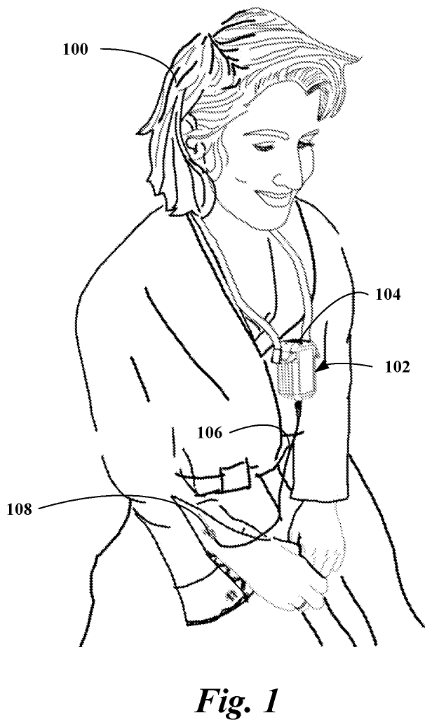

Turning now to FIG. 1, this figure shows a patient 100 receiving photonic infusion therapy via an intravenous light infusion apparatus 102. Apparatus 102 includes compact light engine 104, which generates light at therapeutically-useful frequencies. The frequencies employed will depend on the type of therapy desired. Light from compact light engine 104 passes into and through optical waveguide 106 to vascular access device 108, to infuse the therapeutic light energy into the blood of patient 100.

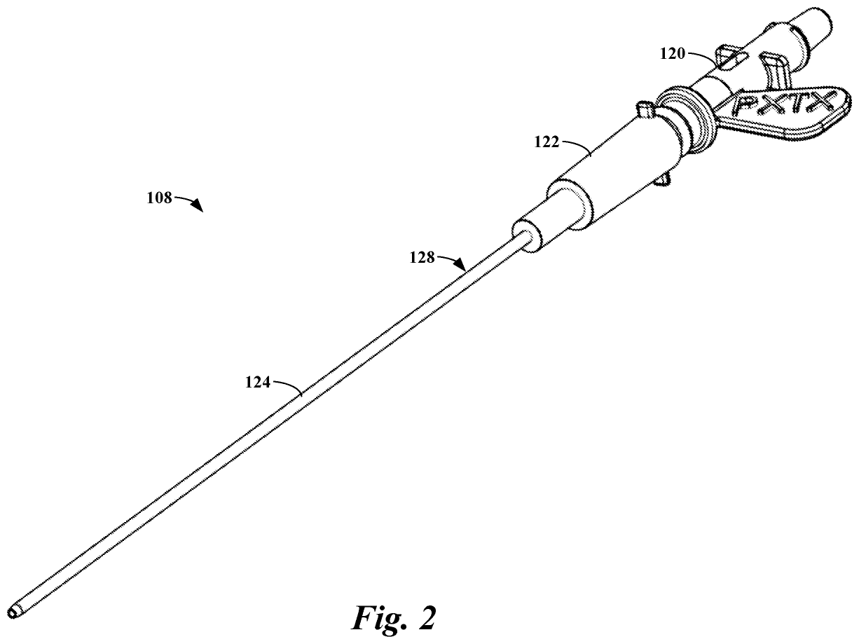

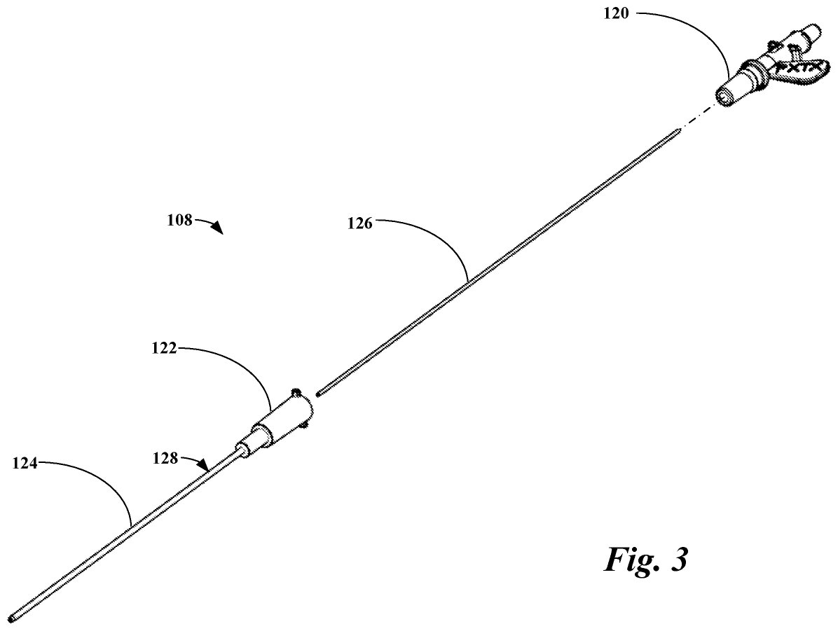

FIGS. 2 and 3 show the vascular access device 108 in an assembled and exploded view, respectively. As seen in these figures, vascular access device 108 includes interface 120, adapter 128, and fiber optic 126. The adapter 128 is further comprised of the adapter housing 122 and adapter lumen 124. Certain details of these components are disclosed below.

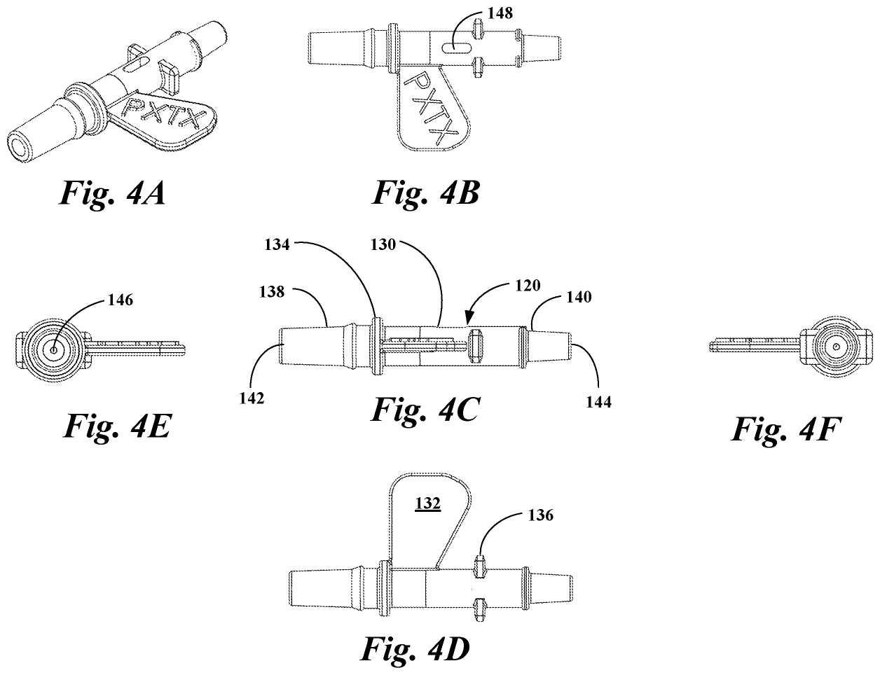

Turning now to FIGS. 4A-4F, interface 120 includes various geometric features, including generally-cylindrical body 130, wing 132, anterior ridge 134, posterior tabs 136, anterior conical surface 138, posterior conical surface 140, front surface 142 and rear surface 144. A generally-cylindrical aperture 146 runs through interface 120. Additionally weld slot 148 extends from the outside of cylindrical body 130 and into cylindrical aperture 146. In certain embodiments, the interface 120 is made of medical grade, radiation resistant plastic. In a preferred embodiment, 120 is made of injection molded plastic with radiation resistance sufficient to endure gamma irradiation sterilization, having biocompatible characteristics compliant with human blood contact for a period of time no less than the intended duration of use and according to ISO 10993 standards, and that is transparent or translucent to allow visualizing the passage into and out of the interface body 120 of fluids and other materials as may be the case in some embodiments as well as allowing certain wavelengths of light, for example UVA1 or BLUE, to pass through with minimal attenuation for the purposes of curing a photosensitive adhesive contained therein. One such example of material is Makrolon polycarbonate Rx2350 with blue tint, available from PolyOne.

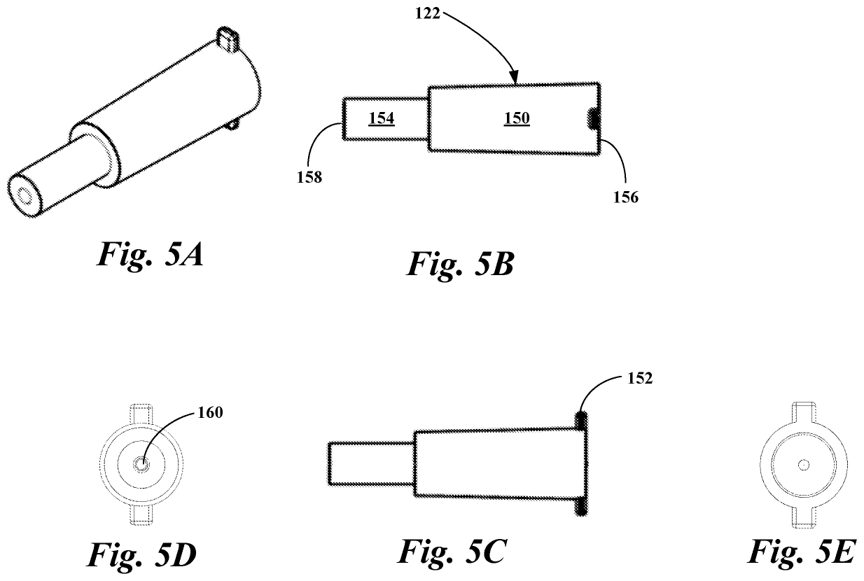

Turning now to FIGS. 5A-5E, adapter housing 122 includes various geometric features, including generally-conical rear section 150 having a pair of anterior tabs 152 disposed thereon and extending radially in opposite directions. Generally-cylindrical front section 154 extends forward from the front of rear section 150. Adapter 122 is bounded by rear surface 156 and front surface 158. A generally-cylindrical aperture passes through adapter 122. From front surface 158 and continuing the diameter of cylindrical aperture 160, adapter lumen 124 extends to a variety of lengths and is constructed from a variety of materials. Lumen 124 may contain a single channel therethrough, or may contain multiple channels. In one preferred embodiment the adapter 128 is a single channel 20 gauge×8 cm or 10 cm midline with a FEP lumen 124.

One should appreciate that the devices described herein provide a simple and direct means of irradiating blood and other body fluids, without the hazards associated with removal and return of fluid volumes and without the possibility of accidental transfer of potentially contaminated fluids between individuals. In addition, isolation of the optical interface from such fluids insures optimal and consistent transmission of light from the light source to the waveguide within the individual undergoing treatment, thereby providing consistent and reproducible irradiation.

The design shown in the attached drawing figures provides a number of advantages. It includes a side tab for ergonomic grip and branding. It includes a push-ring adjacent to the grip tab. The nozzle shape is designed for good press-fit into standard adapters, for example catheters. There is a space between the nozzle 138 ID and the OD of the fiber optic 126, to enable visualizing flash upon proper insertion into catheter. In certain embodiments, the assembly utilizes a high-tack UV(A)-cure medical grade adhesive to prevent adhesive migration within the spaces between the OD of the optical waveguide 126 and the walls of the cyclindrical aperture 160 where the weld slot 148 intersects, both during application and during cure period of adhesive. The weld slot 148 is intended to allow the application of adhesive from outside of the interface 120 to secure the optical fiber 126 within. The integrity of the adhesive must not be damaged by materials, fluids, and normal where and tear. The adhesive must maintain its function after exposure to gamma irradiation sterilization and as part of the finished assembly be compliant with human blood contact for a period of time no less than the intended duration of use and according to ISO 10993 standards. One such adhesive is: AB9112 two part epoxy manufactured by Fiber Optic Center inc. The weld slot and the concept of adhesive migration is an improvement over traditional means of securing one or more components within an assembly. Especially for a disposable product oriented business model, component cost is a major driving factor when considering the viability of any such venture. This inventive concept reduces adhesive migration away from the intended application site(s), which migration may possibly frustrate other assembly features or obscure optical surfaces. Additionally, such adhesive means prevent obscuring of optical surfaces from adhesive off-gassing common to adhesives such as cyanoacrylate.

This device is designed to reduce assembly time and cost, component cost, assembly error and inspection time. In certain configurations, this design may increase manufacturing capacity by 3 to 4 times over other designs. This device can be stored and shipped in a smaller package than prior designs. In certain embodiments, this device can be sterilized in bulk.

In certain embodiments, the device adapter 128 is a 20 gauge×8 cm or 10 cm midline. The device can, in certain configurations, stay indwelling continuously for up to 29 days. A physician can insert the apparatus of the present invention into a patient's arm, and the patient can wear and treat at home with it in for that period of time. A one-way valve can be included in the midline device to allow for administration and removal of fluids. In certain embodiments, the device uses FT600UMT fiber 126.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A vascular access device comprising:
an interface having a principal axis, a first end and a second end;
an adapter, having a principal axis, a first end and a second end, wherein the first end of the adapter is secured to the second end of the interface in such manner that the principal axis of the adapter is aligned to the principal axis of the interface; and
a fiber optic, having a principal axis, disposed within the adapter in such manner that the principal axis of the fiber optic is aligned to the principal axis of the adapter.

2. The vascular access device of claim 1, wherein the adapter further comprises an adapter housing and an adapter lumen attached to the adapter housing.

3. The vascular access device of claim 1, wherein the adapter further comprises a generally-cylindrical aperture passing therethrough.

4. The vascular access device of claim 1, wherein the adapter further comprises a generally-conical rear section.

5. The vascular access device of claim 1, wherein the interface further comprises a generally-cylindrical body having a generally-cylindrical aperture running therethrough.
